(19) United States
(12) Patent Application Publication
(10) Pub. No.: US 2021/0134449 A1
(43) Pub. Date: May 6, 2021

(54) ACUTE MEDICAL CARE SYSTEM

(71) Applicant: EMOPTI, INC., Brookfield, MI (US)

(72) Inventors: Edward BARTHELL, Mequon, WI (US); Robert HEDGCOCK, Delafield, WI (US); Jared RUFER, Hartland, WI (US); Timothy FISCHER, Wauwatosa, WI (US)

(21) Appl. No.: 17/149,309

(22) Filed: Jan. 14, 2021

Related U.S. Application Data
(63) Continuation of application No. 15/796,394, filed on Oct. 27, 2017, now abandoned.
Application No. 15/796,394 is a continuation of application No. PCT/US2016/063390, filed on Nov. 22, 2016.
(60) Provisional application No. 62/259,970, filed on Nov. 25, 2015.

(51) Int. Cl. G16H 40/20; G06N 20/00; G06Q 10/10; G16H 10/40; G16H 70/20; G16H 80/00; G16H 10/60; G06Q 10/06; G16H 20/40; G16H 50/70; G16H 40/63

(52) U.S. Cl. CPC G16H 40/20; G06N 20/00; G06Q 10/103; G16H 10/40; G16H 70/20; G06Q 10/10; G16H 10/60; G06Q 10/0631; G16H 20/40; G16H 50/70; G16H 40/63; G16H 80/00

(57) ABSTRACT

A server center controls operation across one or more command centers that are communicatively networked with one or more medical care settings. Devices are deployed at each medical care setting and at the command center(s) and a control device at the server center facilitates digital consultation sessions between a patient and/or local care provider and command center staff via the devices. The control device also facilitates communication between the command center staff and the care setting data center to update the electronic medical record for a patient and to enter specific patient instructions. The control device also prioritizes patients across one or more care settings and presents a prioritized patient queue to command center staff. The control device also provides command center staff with suggested tests and treatments based on an analysis of patient information. The control device also provides patient admission prediction information to each care setting.

### CROSS-REFERENCE TO RELATED APPLICATION

This application is a continuation of U.S. patent application Ser. No. 15/796,394, filed on Oct. 27, 2017, which is a continuation of International Patent App. No. PCT/US2016/063390, filed on Nov. 22, 2016, which claims priority to U.S. Patent Application No. 62/259,970, filed on Nov. 25, 2015, which are all hereby incorporated herein by reference as if set forth in full.

### BACKGROUND

### Field of the Invention

The present invention is generally related to acute medical care system management and is more specifically related to decreasing delays in testing and treatment, improvement in patient throughput, and accurate and timely prediction of the need for an emergency room patient to be admitted as a hospital observation patient or inpatient.

### Related Art

Conventional acute care facilities are often very busy and inadequate acute medical care performance is often the consequence of overcrowding. Specific results of overcrowding include prolonged waiting times, variability of clinical outcomes, low patient satisfaction, and less desirable economic results for the hospital. Conventional solutions to acute medical care facility overcrowding typically require some combination of adding more patient care rooms and adding more medical and administrative staff. However, these solutions are costly and labor intensive and often times do not adequately address the fundamental patient throughput problems. Specifically, a significant factor causing inadequate acute medical care performance is the isolation of each single acute medical care setting.

For example, a first isolated medical care setting may be a first emergency department and the first emergency department may be staffed with ten physicians on a particular Saturday evening in accordance with the most accurate and up-to-date information available about the expected number of patients that will visit the first emergency department on that particular Saturday evening. However, the particular Saturday evening may be mercifully slow such that only half of the expected number of patients actually visited the first emergency department. Accordingly, the overstaffing of the first emergency department as an isolated single acute medical care setting results in extremely poor efficiency of physician use on that particular Saturday evening. In contrast, a second isolated medical care setting may be a neighboring second emergency department, which may only be staffed with five physicians for the particular Saturday night based on the most accurate and up-to-date information available about the expected number of patients that will visit the second emergency department on that particular Saturday evening. However, the second emergency department may actually receive visits from double the number of anticipated patients, resulting in both very slow treatment of patients with acute medical conditions and very hastily reached medical care decisions when a patient does finally receive a consultation with a physician. Accordingly, the understaffing of the first emergency department as an isolated single acute medical care setting results in extremely inefficient treatment of patients on that particular Saturday evening.

Therefore, what is needed is a system and method that overcomes these significant problems found in the conventional systems as described above.

### SUMMARY

Accordingly, described herein are systems and methods that successfully address single setting acute medical care facility crowding without the drawbacks imposed by conventional solutions. The system includes a server center that controls operation across one or more command centers that are communicatively linked to one or more acute medical care settings and their corresponding data centers. In an acute medical care setting, one or more patient and one or more local care provider interface devices are deployed. In a command center, one or more command center staff interface devices are deployed. A control device at the server center facilitates audio/video/text consultation sessions among a patient and local care provider located at an acute medical care setting and a command center staff located at a command center. The control device at the server center also facilitates communication between the command center and the hospital data center such that an electronic medical record (“EMR”) for a patient can be viewed by and updated by a command center staff at the command center and specific patient testing and treatments can be ordered by a command center staff at the command center. The communication between the command center and the hospital data center also allows the command center to provide admission prediction information to the hospital to facilitate improved readiness for timely admission of patients requiring admission to the hospital. The control device also prioritizes patients across multiple acute medical care settings based on need and additionally provides command center staff with suggestions for tests and treatments based on an analysis of patient information obtained from the patient's EMR and/or clinical messages (e.g., Health Level 7 (“HL7”) messages). The control device also analyzes individual and aggregate patient data to provide anonymized statistical information regarding acute medical care setting performance such as the average amount of time patients wait to see a local care provider or a command center staff and which tests and treatments are ordered for different patient types/conditions.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is a network diagram illustrating an example system for improved acute medical care across multiple care settings according to an embodiment of the invention;

FIG. 2 is a network diagram illustrating an example system for improved acute medical care across multiple departments of a hospital setting according to an embodiment of the invention;

FIG. 3 is a network diagram illustrating an example system for improved acute medical care having patient, local care provider, command center staff and data center interface devices coordinated by a server center control device according to an embodiment of the invention;

FIG. 4 is a block diagram illustrating an example control device for implementing improved acute medical care according to an embodiment of the invention;

FIG. 5A is a block diagram illustrating an example real time command center staff display according to an embodiment of the invention;

FIG. 5B is a block diagram illustrating a settings region of the example real time command center staff display of FIG. 5A according to an embodiment of the invention;

FIG. 6 is a flow diagram illustrating an example process for conducting a consultation session between a command center staff and a patient according to an embodiment of the invention;

FIG. 7 is a flow diagram illustrating an example process for establishing a digital consultation session according to an embodiment of the invention;

FIG. 8 is a flow diagram illustrating an example process for determining a hospital admission estimate for a patient according to an embodiment of the invention; and

FIG. 9 is a block diagram illustrating an example wired or wireless processor enabled device that may be used in connection with various embodiments described herein.

### DETAILED DESCRIPTION

Certain embodiments disclosed herein provide for improved acute medical care management using a command center staffed with personnel capable of consulting with patients across multiple emergency rooms. The system also provides accurate estimates of patient admissions so the hospital can more efficiently admit patients from the acute medical care facility to the hospital. For example, one method disclosed herein prioritizes patients across multiple care settings and establishes consultation sessions between command center staff and the highest priority patient in order to more rapidly address patient needs when patient volume exceeds available staffing levels at the various care settings. After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

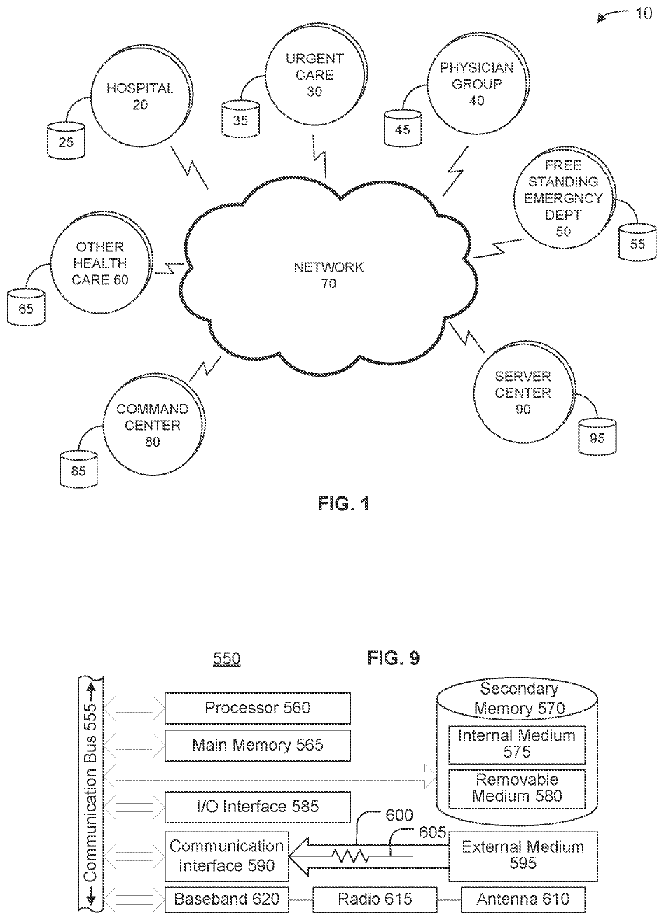

FIG. 1 is a network diagram illustrating an example system 10 for acute medical care across multiple care settings that include one or more of the following: one or more hospitals 20, one or more urgent care facilities 30, one or more independent provider groups 40, one or more free standing emergency departments 50 and one or more other health care settings 60 according to an embodiment of the invention. Although there may be plural instances of any of the illustrated settings 20, 30, 40, 50 and 60, the network 70, the command center 80 and the server center 90, for ease of discussion, the following description will discuss them in the singular except where helpful to discuss them in the plural.

In the illustrated embodiment, the hospital 20, urgent care facility 30, independent provider group 40, free standing emergency department 50 and other health care setting 60 are each communicatively coupled with a command center 80 and a server center 90 via the network 70. In one alternative embodiment, the command center 80 is co-located with a care setting and communicatively coupled with the elements of the care setting via a network such as network 70. Each of the illustrated settings 20, 30, 40, 50 and 60, the command center 80 and the server center 90 include one or more processor enabled devices as later described with respect to FIG. 9, each of which is configured with one or more data storage areas 25, 35, 45, 55, 65, 85 and 95, respectively. The data storage areas may be implemented as a single data storage device or as multiple data storage devices or as a remote data storage device or the like.

In practice, the server center 90 facilitates communication between the illustrated settings 20, 30, 40, 50 and 60 and the command center 80. The server center 90 may also facilitate communication directly between two or more of the illustrated settings 20, 30, 40, 50 and 60. In one embodiment, the server center 90 establishes digital consultation sessions between command center staff at the command center 80 and a patient at one of the illustrated settings 20, 30, 40, 50 and 60 (e.g., an emergency department operated by the hospital 20 or a free standing emergency department 50). The server 90 also establishes digital consultation sessions between command center staff at the command center 80 and a local care provider at one of the illustrated settings 20, 30, 40, 50 and 60. As used herein, “command center staff” may include a physician, physician assistant, nurse, nurse practitioner, lab technician, medical assistant, other clinical support staff, resident medical student, other student, trainee, and the like. Additionally, as used herein, “local care provider” may include a physician, physician assistant, nurse, nurse practitioner, lab technician, medical assistant, other clinical support staff, resident medical student, other student, trainee, and the like.

By way of the digital consultation sessions between command center staff at the command center 80 and the local care provider and/or the patient at one of the illustrated settings 20, 30, 40, 50 and 60, command center staff is able to treat the patient and order additional tests and/or treatment protocols as necessary by way of making entries directly into the patient's electronic medical record (“EMR”), which is also facilitated by the server center 90. Accordingly, the command center 80 is advantageously able to reduce the workload of local care providers who are physically located at one of the illustrated settings 20, 30, 40, 50 and 60 and, for example, reduce crowding across a plurality of settings operated by a plurality of hospitals and hospital systems and other entities.

In one embodiment, the command center 80 is able to provide a comprehensive perspective across all of the illustrated settings 20, 30, 40, 50 and 60. The comprehensive view advantageously provides command center staff with a complete overview of patient statuses across all care settings and provides command center staff the ability to drill down into each individual care setting to view comprehensive patient statuses within a single care setting. The comprehensive view also provides command center staff the ability to drill down into a single location (e.g., a single emergency department of a hospital system) to view comprehensive patient statuses within a single care setting. This capability advantageously allows utilization of the command center 80 to increase local care provider utilization, dynamically assist with patient throughput, provide direction on where patients should go, provide medical oversight, reduce “door-to-doc” time, provide specialty care, provide recommendations to optimize resource allocation throughout the acute care system, orchestrate the allocation of human and other resources and other activities to optimize patient flow and the delivery of care through the acute care system, and assess the performance of a medical student or trainee.

Utilization of the command center 80 may also facilitate care transitions of one or more patients at any one of the illustrated settings 20, 30, 40, 50 and 60 to any other of the illustrated settings 20, 30, 40, 50 and 60—whether there is an existing administrative relationship between the two settings or not. For example, a patient who is presently at an urgent care 30 facility that has no administrative relationship with hospital 20 and who is also being monitored by command center staff at the command center 80 can be transitioned over to the emergency department at hospital 20 via the command center 80. The command center 80 can provide the hospital 20 with the necessary information to prepare for the arrival of the patient. In one embodiment, the command center 80 can update an electronic medical record for the patient at the hospital 20 before the patient arrives at the hospital 20 to facilitate a more smooth transition of the patient from the urgent care 30 setting to the hospital 20 setting. In one embodiment, a uniform user interface is provided for command center staff to enter information into various different electronic medical record systems that may be employed by the various different illustrated settings 20, 30, 40, 50 and 60. The uniform user interface improves accuracy and reduces the time required for command center staff to enter information into electronic medical records.

An additional advantage provided by the system 10 includes the ability to provide command center staff at the command center 80 with suggested tests and/or treatments based, e.g., on the patient's chief complaint as initially recorded upon entry to one of the illustrated settings 20, 30, 40, 50 and 60 and subsequently recorded and/or augmented in connection with subsequent caretaker interactions with the patient. An additional advantage is that the system can track compliance with care standards based on recommendations provided by the system for the patient corresponding to the patient situation and medical and demographic information. Another advantage provided by the system 10 includes the ability to provide a hospital admission prediction and an estimated time of hospital admission for each patient being monitored by the system. For example if the admission prediction is over 80% then an estimated time of day for hospital admission may also be provided. Yet another advantage provided by the system 10 is analysis of anonymized patient data to provide statistics and metrics about the performance of any of the illustrated settings 20, 30, 40, 50 and 60. For example, the system can provide statistics related to the average time for a patient to see a local care provider.

In one embodiment, command center staff at the command center 80 and local care providers at any one of the illustrated settings 20, 30, 40, 50 and 60 have access to information and have the ability to initiate actions. For example, through the system 10, command center staff at the command center 80 and local care providers at any one of the illustrated settings 20, 30, 40, 50 and 60 have access to patient medical record information, patient satisfaction information, current setting demographics such as the number and skill sets of local care providers and the number of current open beds. Additionally, command center staff at the command center 80 and local care providers at any one of the illustrated settings 20, 30, 40, 50 and 60 also have the ability through the system 10 to order tests, enter data and/or instructions into a patient's electronic medical record, provide information to patients and families to facilitate the discharge of patients, and view dashboards that show patient flow activity, metrics, and other situational awareness information about a patient or the setting.

In a simple embodiment, hospital setting 20 is a single hospital at a single physical location with a single emergency department and a data center resident at the single physical location. In a more complex embodiment, hospital setting 20 includes a plurality of hospitals with different physical locations with a plurality of emergency departments with different physical locations (co-located with a hospital or free-standing) and one or more remote data centers that process data for the entire hospital system that comprises the hospital setting 20.

In one embodiment, urgent care setting 30 includes one or more separate free standing physical locations and is configured to care for patients that present with acute problems and without a previous appointment to be seen by a local care provider. The urgent care setting 30 may also be co-located with an outpatient clinic facility. In one embodiment, urgent care setting 30 may also be co-located with a hospital setting 20.

In one embodiment, independent provider group setting 40 includes one or more separate free standing physical locations and is configured to care for patients that present with mild to acute problems, with or without a scheduled appointment to be seen by a local care provider. The independent provider group setting 40 may include sophisticated emergency department acute medical care capabilities. The independent provider group setting 40 may also be co-located with a hospital setting 20 while being separately operated from an administrative standpoint.

In one embodiment, the free standing emergency department setting 50 includes one or more separate free standing physical locations and is configured to care for patients that present with acute problems and without a previous appointment to be seen to be seen by a local care provider. The free standing emergency department setting 50 may also be co-located with an outpatient clinic facility or other medical service providers.

In various embodiments, other health care setting 60 may include a medical transport vehicle (e.g., ambulance, helicopter, etc.), a home, a school, an office building or other commercial building (e.g., a sports stadium), a prison, a long term care facility, an inpatient acute care facility (e.g., intensive care unit, burn unit, etc.), a teaching hospital, an academic medical center, a skilled nursing facility or other remote health care facility. As previously discussed, there are significant benefits that result from connecting the command center 80 to the various other health care settings 60, such as increasing local care provider utilization, dynamically assisting with patient throughput, providing direction on where patients should go, reducing transport cost, providing medical oversight, reducing “door-to-doc” time, providing specialty care, providing recommendations to optimize resource allocation throughout the acute care system, providing recommendations to orchestrate the allocation of human and other resources, providing recommendations to orchestrate other activities to optimize patient flow and the delivery of care through the acute care system, and assessing the performance of a medical student or trainee.

In one embodiment, the command center 80 is a free standing physical location that is separate from any of the various settings 20, 30, 40, 50 and 60 and separate from the server center 90. Such a free standing physical location may be in a commercial building or a residence. In an alternative embodiment, the command center 80 may be integrated with any of the various settings 20, 30, 40, 50 and 60 or integrated with the server center 90. In one embodiment, the command center 80 may be integrated with the server center 90 and the combined command center 90 and server center 90 may be integrated with any of the various settings 20, 30, 40, 50 and 60.

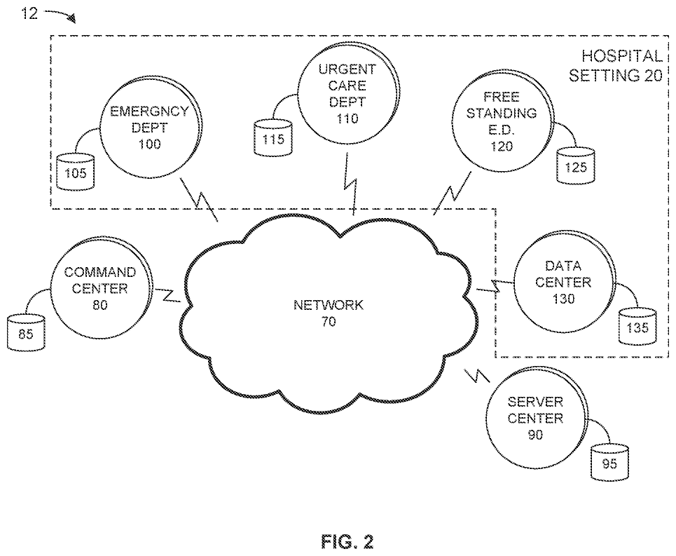

Turning now to FIG. 2, the following detailed description will focus on an embodiment having a single hospital setting. However, it should be understood that the following description and its corresponding drawings represent only one embodiment of the invention and therefore the following description and its corresponding drawings are merely representative of the subject matter which is broadly contemplated by the present invention.

FIG. 2 is a network diagram illustrating an example system 12 for improved acute medical care management across multiple departments 100, 110 and 120 of a hospital setting 20 according to an embodiment of the invention. In the illustrated embodiment, the hospital 20 has an emergency department 100, an urgent care department 110, a free standing emergency department 120 and a data center 130. The emergency department 100 is part of the physical hospital location while the free standing emergency department 120 is separate from the physical hospital location. Each of the departments within the hospital setting 20 are communicatively networked to each other and also communicatively networked to the command center 80 and the server center 90 via the network 70. The hospital setting 20 may also include various additional and/or alternative departments such as an affiliated or independent provider group or other health care facility.

In alternative embodiments, the hospital 20 can have one or more instances of each of the various departments 100, 110 and 120 and the data center 130 can be co-located with any of the illustrated departments 100, 110 and 120 or it can be separate. In another alternative embodiment, several hospital settings can be networked to one or more command centers 80 and server centers 90 via the network 70. In a simple embodiment, the hospital setting 20 comprises a single emergency department 100 and the data center 130 is in the same physical location.

In the illustrated embodiment, each of the various departments 100, 110, 120 and also the data center 130 include one or more processor enabled devices (e.g., such as the device later described with respect to FIG. 9) that are configured with one or more data storage areas 105, 115, 125 and 135, respectively. Each of the various data storage areas may be implemented as a single data storage device or as multiple data storage devices.

In practice, the server center 90 facilitates communication between the various departments 100, 110 and 120 of the hospital setting 20 and the command center 80. The server center 90 may establish digital consultation sessions between command center staff at the command center 80 and a local care provider at one of the departments 100, 110 or 120 of the hospital setting 20. The server center 90 may also establish separate digital consultation sessions between command center staff at the command center 80 and a patient at one of the departments 100, 110 or 120 of the hospital setting 20. By way of these sessions between the command center staff at the command center 80 and the local care providers and patients at the various departments 100, 110 or 120 of the hospital 20, command center staff are able to treat the patient. Additionally, command center staff are able to order tests and/or implement treatment protocols as necessary by way of making entries directly into the patient's EMR, which is facilitated by communication between the server center 90 and the data center 130. Specifically, the command center staff at the command center 80 can enter orders into the patient's EMR at the command center 80 and the server center 90 facilitates the order being entered into the patient's EMR at the hospital data center 130 so that action can be taken by a local care provider at the hospital 20.

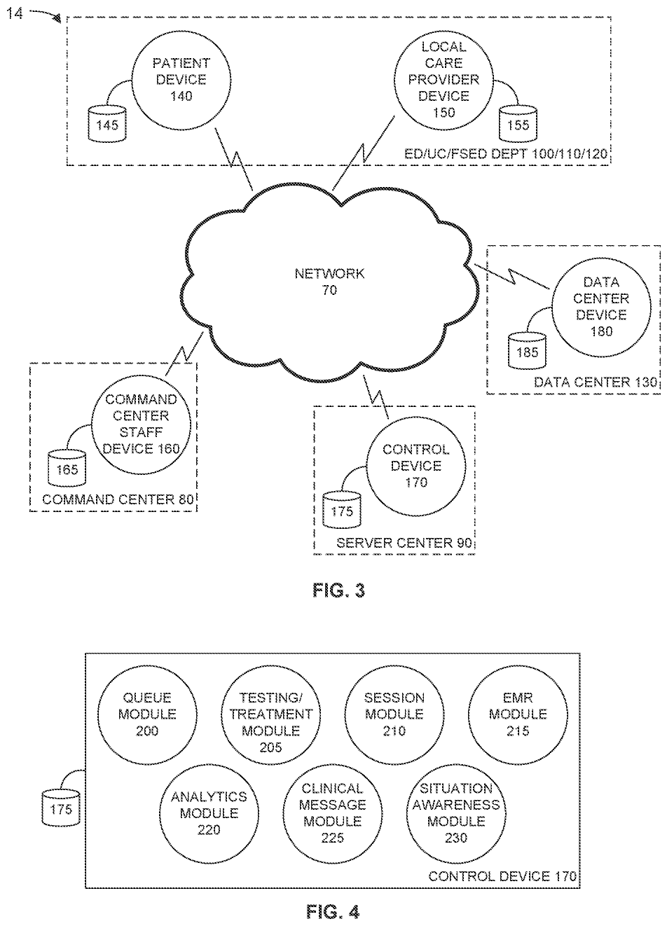

FIG. 3 is a network diagram illustrating an example system 14 for improved acute medical care management having patient device 140, a local care provider device 150, a command center staff device 160 and a data center device 180. Each of the devices 140, 150, 160 and 180 is configured to present a user interface to its respective user and communication between the devices 140, 150, 160 and 180 is coordinated by a server center control device 170 according to an embodiment of the invention. In some embodiments, a local care provider device 150 may include any type of electronic medical care device such as a stethoscope, otoscope, examination camera, medical imaging device, heart rate monitor, flash light, life support equipment, medical monitors and the like. In the illustrated embodiment, the system 14 comprises a patient device 140 and a local care provider device 150 at any of the various departments 100, 110 and 120 previously described with respect to FIG. 2. Additional devices (not shown) may also be present at the various departments, e.g., a triage device or any other devices involved in patient care and patient management and/or any other devices capable of generating clinical messages (e.g., HL7 messages) that can be delivered to the server center 90 via the network 70. It should be noted that HL7 is used herein with respect to the communication of hospital related clinical messages but the system is capable of using a variety of different clinical messaging formats and other messaging formats and therefore the system is not limited to the use of HL7 nor is the system required to use HL7.

In one embodiment, the patient device 140 and local care provider device 150 can also be implemented as a single physical device. The system 14 also comprises a command center staff device 160 at a command center 80, a control device 170 at a server center 90 and a data center device 180 at a hospital data center 130. In one embodiment, the command center 80 and the server center 90 can be co-located. The hospital data center 130 and one or more of the departments 100, 110 and 120 can also be co-located as previously discussed.

Each of the devices 140, 150, 160, 170 and 180 can be implemented as processor enabled devices such as the device later described with respect to FIG. 9. Each of the devices 140, 150, 160, 170 and 180 are configured with respective data storage areas 145, 155, 165, 175 and 185. Each of the devices 140, 150, 160, 170 and 180 are also configured to communicate with other devices via the network 70. In one embodiment, devices that are near each other (e.g., a patient device 140 and a local care provider device 150) may be configured to communicate with each other via alternative wired or wireless means such as Bluetooth®.

The patient device 140, local care provider device 150 and command center device 160 may each include one or more of a camera, microphone, speaker and display in order to establish and maintain a real-time audio-visual consultation session between command center staff at the command center 80 and a patient and/or a local care provider at the various departments 100, 110 and 120. The consultation session is established and facilitated by the control device 170 and implemented via the network 70. Once the session is established, the audio and visual data of the consultation session may be transmitted over the network 70 directly between the patient device 140 or local care provider device 150 and the command center staff device 160. Alternatively, once the session is established, the audio and visual data of the consultation session may be transmitted over the network 70 indirectly between the patient device 140 or local care provider device 150 and the command center staff device 160 with the control device 170 functioning as a pass-through or switching mechanism for all of the session data. One advantage of indirect session communication is that the control device 170 may optionally store all of the session data for real-time or subsequent review and/or analysis.

The command center device 160 is also configured for direct or indirect (via control device 170) communication with the data center device 180 in order to present an EMR of a patient on the display of the command center device 160. Advantageously, communication between the command center device 160 and the data center device 180 (whether via the control device 170 or not) allows command center staff to enter information into the EMR of the patient for storage by the data center device 180 in a data storage area 185 of the hospital data center 130. Accordingly, orders entered by command center staff at the command center 80 initiate actions at the various departments 100, 110 and 120 to carry out any testing and treatment protocol as instructed by command center staff at the command center. In one embodiment, the command center device 160 comprises a plurality of displays for presentation of information to command center at the command center 80. In an alternative embodiment, the command center device 160 comprises a single display that is logically divided into a plurality of sections for presentation of information to command center staff at the command center 80.

The data center device 180 is communicatively coupled with other systems within the hospital setting such as the laboratory, radiology, billing and admissions and consequently the command center device 160 (e.g., via the control device 170) is able to initiate orders for laboratory tests, X-RAYs or other imaging, etc. The control device 170 is also able to provide admission estimates to the hospital setting via communication between the control device 170 and the data center device 180.

FIG. 4 is a block diagram illustrating an example control device 170 for implementing improved acute medical care management according to an embodiment of the invention. The control device 170 is a processor enabled device such as the device later described with respect to FIG. 9. In the illustrated embodiment, the control device 170 comprises a queue module 200, a suggested testing/treatment module 205, a session module 210, an EMR module 215, an analytics module 220, a clinical message module 225 and a situation awareness module 230.

The queue module 200 is configured to establish a queue of patients in various settings (e.g., emergency department, urgent care, long term care facility, etc.) waiting for consultation with command center staff. In one embodiment, the queue module 200 establishes a queue of all patients that have checked into the various care settings. In an alternative embodiment, the queue module 200 establishes a queue of only those patients that have checked into the care setting and have also been identified by a local care provider as a candidate for a session with command center staff at the command center. The queue module 200 is advantageously configured to prioritize patient queues. A patient queue can be for a single department or for a plurality of departments of a single hospital setting or for a plurality of departments for a plurality of settings.

In one embodiment, the control device 170 is configured to receive clinical messages (e.g., HL7 messages) from the hospital via the network 70 and the control device 170 is further configured to parse the HL7 messages to identify patients that have checked into one of the various departments. For example, an admission/discharge/transfer (“ADT”) message is a form of HL7 message and the queue module 200 is configured to analyze the ADT message and obtain initial patient information from the ADT message and add the patient to a particular department's patient queue. The queue module is configured to maintain a plurality of separate patient queues, one for each of a plurality of separate departments that may belong to one or more settings (e.g., a hospital setting, a long term care facility setting, a mobile transport vehicle setting, an urgent care setting, etc.).

In addition to maintaining a queue of patients, the queue module 200 is configured to prioritize the patients in each patient queue relative to each other. For example, the queue module 200 may analyze patient information parsed from a plurality of HL7 messages to determine a rank for each individual patient and then prioritize the patients in the patient queue according to rank. In one embodiment, patients with the same rank can be prioritized relative to each other by a secondary metric such as wait time so that between two patients of equal rank, the patient waiting longer has the higher priority. Alternative prioritization techniques may also be employed.

In addition to ranking the patients in each department's patient queue, the queue module is also configured to globally prioritize all patients across all department patient queues relative to each other. Similar prioritization techniques may be employed as described above. Advantageously, the global prioritized patient queue allows the control device 170 to present to command center staff at the command center 80 a globally prioritized patient queue to allow the highest priority patient across a plurality of departments and a plurality of settings to more quickly receive a consultation with command center staff at the command center. Additionally, the queue module 200 is configured to provide the global patient queue and the individual department patient queues to the command center for presentation on the display of one or more command center staff devices.

The testing and treatment module 205 is configured to analyze patient information parsed from a plurality of clinical messages to determine a suggested treatment for each individual patient. For example, the suggested treatment module 205 is configured to parse the patient's chief complaint from the ADT message and any further HL7 messages including a chief complaint and analyzes the chief complaint data to determine a treatment code. The treatment code can, for example, be part of a code set such as the frontlines of medicine code set. It should be noted that other code sets and algorithms could be used. Advantageously, the treatment code can be translated into a suggested treatment protocol that can be provided by the suggested treatment module 205 via the network 70 and presented on the display of a command center staff device 160 at the command center 80.

The testing and treatment module 205 is also configured to present information on the display of command center staff device 160 at the command center 80. For example, the testing and treatment module 205 may present suggested test and treatment information as described above and additionally present related testing and treatment protocol information, reference materials, contact information, and other information helpful to the command center staff when considering treatment of a patient. The testing and treatment module 205 may also present a search box on the display of command center staff device 160 at the command center 80 to allow the command center staff to search for and display additional information related to patient care or otherwise.

The session module 210 is configured to establish a session between a patient device 140 and the command center staff device 160 via the network 170. Additionally, the session module 210 is configured to establish a session between a device at one of the various departments 100, 110 or 120 (e.g., local care provider device 150 or patient device 140) and the command center staff device 160 at the command center 80 via the network 70. In various embodiments, a session may include real-time audio and video, audio only, video only, text, or any combination of the above and other elements capable of being communicated between devices via the network 70. The session module 210 may be configured to initiate and hand-off the session for direct communication between devices or initiate and facilitate the session by passing through the communication between devices or some combination of initiation followed by passive monitoring or active participation or no monitoring or participation. Advantageously, the session module 210 may be configured to record and store in a data storage area some portion of the data or all of the data associated with a session for subsequent review and/or analysis.

The EMR module 215 is configured to interface with the data center device 180 and receive information regarding a patient's EMR and present EMR data on the display of command center staff device 160 at the command center 80. The EMR module 215 is also configured to receive input from command center staff via the command center staff device 160 at the command center 540 and provide that input to the data center device 180 for entry into the patient's EMR maintained by the hospital data center 130. In one embodiment, the EMR module may receive an indication from the consulting command center staff to accept suggested treatment information provided by the suggested treatment module 205 and presented on the display of command center staff device 160 at the command center 80. In such an embodiment, the suggested treatment module 205 provides the information to the EMR module 215 as an order for patient treatment and the EMR module 215 in turn provides the order to the data center device 180 at the hospital data center 130 for entry into the patient's EMR.

The analytics module 220 is configured to analyze stored information regarding patient care and performance of the various departments for the benefit of the hospital setting. For example, information analyzed can include timestamped HL7 messages to identify the path a patient traveled through the emergency department and hospital system and determine the amount of time allotted to each segment of the overall patient experience at the hospital. Advantageously, the analytics module 220 can determine the extremely valuable “door-to-doc” metric that can be used by a hospital system or an emergency department to evaluate the efficiency of staffing levels and other operational aspects of emergency department management and/or hospital system management.

The analytics module 220 is also configured to provide real-time admission estimates for patients in the various department patient queues. These estimates may be based on chief complaint data from the ADT message and subsequent HL7 messages in addition to suggested treatment information and actual command center staff treatment orders entered into the patient's EMR. Advantageously, an initial admission estimate can be determined by the analytics module 220 and provided to the hospital data center 130 for routing to the appropriate admissions staff. In one embodiment, the analytics module 220 only provides the initial admission estimate to the hospital data center 130 if the estimate exceeds a predetermined threshold, for example 80%. In an alternative embodiment, the analytics module 220 estimates what capability and level of care will be required for admitted patients or expected to be admitted patients. This capability and level of care is referred to as type of admission bed. For example, the types of admission beds in a medical care setting are generally categorized into three types: (1) an intensive care unit bed; (2) a telemetry bed; and (3) a general medical/surgical bed. Some medical care settings may use alternative terms to describe their types of admission beds. Additionally, the analytics module is configured to update the initial admission estimate over time as new information regarding treatment of the patient is received, for example by way of HL7 messages or command center staff entries into the patient's EMR. Advantageously, the analytics module can provide revised admission estimates to the hospital data center 130 over time or provide a first admission estimate to the hospital data center 130 substantially after the patient has checked into the emergency department—all dependent upon the analytics module 220 analysis of data related to the admission prediction.

Additionally, the analytics module 220 is configured estimate a time of admission for each patient. In one embodiment, the estimated time of admission for a patient is only provided to the hospital data center 130 if the admission estimate exceeds a predetermined threshold, for example 90%. Accordingly, in one embodiment, the analytics module 220 is configured to signal to the hospital data center 130 that a patient is likely to be admitted if the admission estimate is 80% or greater and the analytics module 220 is configured to signal to the hospital data center 130 an estimated time of admission if the admission estimate is 90% or greater. Furthermore, the analytics module 220 is configured to continuously update the admission estimate and estimated time of admission as new information regarding the treatment of the patient is received.

In one embodiment, the analytics module 220 is configured to provide an estimated time of admission estimate for individual patients in a plurality of departments across a hospital setting to allow the hospital setting to manage patient admissions and arrange for patient transfers across multiple physical locations of the hospital setting.

In one embodiment, the analytics module 220 is configured to analyze historical patient data and recommend improvements to testing and treatment protocols associated with treatment codes. Advantageously, the recommendations provided by the analytics module 220 can be based on actual orders from command center staff for testing and treatment of actual patients and the actual outcomes of such testing and treatments. For example, the data analyzed by the analytics module 220 can be gleaned from clinical messages. Additional information analyzed by the analytics module 220 can include the presence of additional tests and treatments ordered by the command center staff that were not part of the initial recommendations provided by the suggested treatment module 205. In this fashion, the analytics module 220 can identify improvements and trends in actual testing and treatment of patients having a particular treatment code derived from the patient's chief complaint.

The clinical message module 225 is configured to receive and process clinical messages, for example, HL7 messages. Parsing of HL7 messages may include excising particular fields of data and/or portions of information and translation such data and/or information. For example, the clinical message module 225 may receive the ADT message and parse the patient's chief complaint from the ADT message and translate the chief complaint into a treatment code. Additionally, the clinical message module 225 may receive HL7 messages and parse the messages to identify the time of day for each step in the patient's visit to the emergency department so that a chronological timeline of the patient's visit can be constructed and the “door-to-doc” metric determined for one or more aspects of the patient's visit.

The situation awareness module 230 is configured to analyze patient data and emergency department and hospital data and derive real time analytics that can be presented on a display, for example on a display at the command center to allow command center staff or other consultant (e.g., manager, administrator) to provide recommendations for improved operations at any given setting. In one embodiment, the real time analytics derived by the situation awareness module 230 can be presented in the later described administrative management data section of the command center staff display. Advantageously, the situation awareness module 230 is configured to provide an overview of patient location at any given time as multiple patients transition through the stages of acute care. A significant advantage of providing a real time overview of the patient processing stream is that the overall hospital setting resources and the various department resources can be appropriately assigned and/or reassigned to reduce existing bottlenecks or to prevent anticipated bottlenecks.

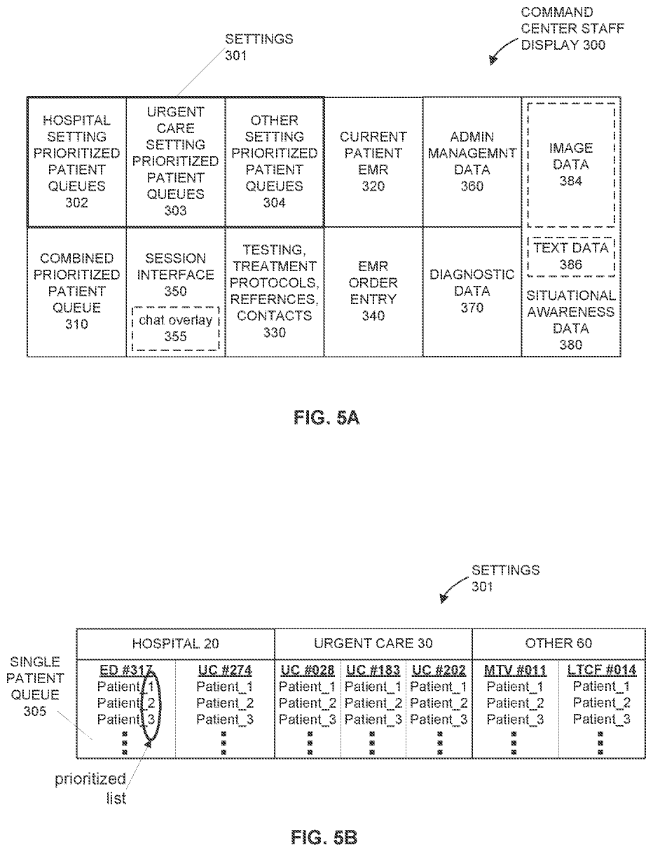

FIG. 5A is a block diagram illustrating an example user interface of a real time command center staff display 300 according to an embodiment of the invention. The user interface 300 may comprise multiple physical display elements or the user interface 300 may be a single physical display element logically divided into multiple sections, or some combination of physical and logical sections of the user interface 300. In the illustrated embodiment, the user interface 300 comprises a settings region 301 that includes a plurality of patient queues 302, 303 and 304. Each of the plurality of patient queues 302, 303 and 304 may be associated with a single department of a hospital setting 20 or they may be associated with departments divided between two or more settings such as a hospital setting 20, an urgent care setting 30 and so forth. In the illustrated embodiment, settings region 301 includes a hospital setting patient queue 302, an urgent care setting patient queue 302 and an other setting patient queue 302.

In the illustrated embodiment, the user interface 300 also comprises a combined prioritized patient queue section 310, a current patient EMR section 320, a suggested testing and treatment section 330, an EMR order entry section 340, a session interface section 350, an administrative management data section 360, a diagnostic data section 370 and a situational awareness data section 380 that includes an image data region 384 and a text data region 386.

The combined prioritized patient queue section 310 advantageously presents the highest priority patients ranked across all of the various settings being managed by the acute medical care system. Additionally, each of the patient queues 302, 303, 304 and 310 may include certain information about each patient such as a patient's vital signs. According to the medical community, vital signs may include a variety of different information about a body's most basic functions. As used herein, vital signs are defined as: heart rate, blood pressure, body temperature, respiratory rate, and oxygen saturation level.

The current patient EMR section 320 is configured to present EMR data for a selected patient with whom the command center staff is presently consulting. The EMR data may include patient family and medical history, vital signs, testing and diagnostic orders, lab results, imaging results, EKG results, previous patient encounter information, previous diagnoses, notes and other information related to a patient and/or the patient's history and the patient's current medical condition and/or needs. In this example, “presently consulting” means that one command center staff has selected the patient in a patient queue and thereby indicated an intent to have a digital consultation session (e.g., a real time audio-visual session) with the patient. Additionally, “presently consulting” may also mean that the command center staff is presently having, or recently finished having a digital consultation session with the patient. In one embodiment, when a command center staff selects a patient from a patient queue, the current patient EMR 320 section and other sections of the specific command center staff display 300 being used by the command center staff that selected the patient are populated with information specific to that patient and the patient is deemed to be selected by the command center staff for consultation. For example, after selection but prior to the digital consultation session, the command center staff may review the EMR of the patient and the suggested treatments in order to prepare for the digital consultation session. Advantageously, the command center staff can review the patient's EMR data in the current patient EMR section 320 prior to establishing the digital consultation session and the command center staff can enter testing and treatment orders in the patient's EMR in the EMR order entry section 340 after the digital consultation session has terminated.

The testing and treatment section 330 is configured to present suggested treatment information for the current patient and may also be configured to present additional information for consideration by the command center staff. The suggested treatment section 330 may also include a search box to allow the command center staff to search medical databases or other resources for information helpful in diagnosis and treatment of the current patient.

The EMR order entry section 340 is configured to receive input from the command center staff. The received input is provided by the control device 170 to the data center device 180 to facilitate entry of the data into the EMR of the patient that is maintained by the hospital data center 30. Advantageously, such entry in the EMR will initiate action at the emergency department to facilitate treatment of the patient in accordance with the command center staff instructions.

The session interface section 350 is configured to present video and/or text and/or other data associated with a current digital consultation session between the command center staff and a patient or a local care provider or other personnel. The session interface section 350 may also be configured to manage text and audio data associated with a current digital consultation session and deliver text data to a chat overlay portion 355 of the user interface 300 and also deliver audio data through one or more audio output devices (not shown) that are proximal to the user interface 300.

In one embodiment, the session interface 350 may include a patient section (not shown) and a separate local care provider section (not shown) and/or a separate chat section 355. The provider may thus be able to simultaneously see both the patient and the local care provider in separate sections of the session interface 350, for example when the patient and the local care provider have separate devices. There may also be a separate chat section 355 that is overlayed on the session interface 350, for example over a portion of the local care provider section to allow the local care provider and the provider to conduct private communication via text during the consultation session between the provider and the patient.

The administrative management data section 360 is configured to present administrative management data and receive input from command center staff. In one embodiment, administrative management data may include performance metrics and other administrative data for a single emergency department or multiple emergency departments of a single hospital system or across a plurality of hospital systems. The administrative management data section 360 may also present additional session requests. For example, if a local care provider desires to have a follow up session with command center staff regarding a particular patient, the patient name (or other moniker) can be presented in the administrative management data section 360 such that receiving a selection of the presented name/moniker causes the control device to populate the patient EMR data into the current patient EMR section 320 and populate test results and other information related to the patient into the diagnostic data section 370 along with populating the other sections of the command center staff display 300. The control device can also establish a follow up digital consultation session between the provider device and the local care provider device to facilitate a discussion between the local care provider and the provider. Additionally, during the follow up digital consultation session, the EMR order entry section 340 can receive new or revised testing and/or treatment orders for the patient that are subsequently delivered to the hospital system data center. Advantageously, the requested follow up sessions listed in the administrative management data section 360 can also be prioritized based on desired criteria.

The administrative management data section 360 may also present alternative session enabling monikers such that when a selection of a session enabling moniker is receive, a digital consultation session is established. For example, a digital consultation session may be established to facilitate remote monitoring of an in-person interaction between a student provider and a patient or a resident provider and a patient for educational purposes. Additionally, a digital consultation session may be established to facilitate remote consultations by command center staff who is an expert in a particular field. Advantageously, any digital consultation session can be recorded and stored in memory for subsequent use for teaching or administrative or other purposes.

The diagnostic data section 370 is configured to present real-time or nearly real time information regarding a patient. For example, electrocardiogram (“EKG”) data, imaging data (e.g., sonogram, xray, etc.), observation data (e.g., vital signs, etc.), and laboratory information. Other types of diagnostic data may also be presented in the diagnostic data section 370. Advantageously, such information can be presented in the diagnostic data section 370 prior to when the data is available in the patient's EMR and therefore the patient may be able to be processed through the emergency department by a consulting provider more quickly.

The situational awareness data section 380 is configured to present image data in the image data section 384 and text data in the text data section 386 to assist command center staff. The situational awareness information provides the command center staff with the information and insights that would be available if the command center staff person were on-site at an individual facility, and in an alternative embodiment provides a unique meta-level view of data originating from multiple facilities simultaneously. This type of broader overview can advantageously inform command center staff decision makers with respect to operational decision making to improve the efficiency of emergency department, hospital, or hospital system operations, including but not limited to resource load balancing, prioritization of telemedicine interventions and other decisions that are not possible when viewing data from only a single facility. For example, real time camera views into the waiting rooms of the ED or Urgent Care facilities being supported from the command center rapidly provide information about the volume of patient flow. Additional examples include one or more user interface dashboards presented on the display of one or more the command center devices that identify the number of patients at different stages of emergency department care at multiple facilities and/or provide visibility into available hospital resources (e.g., local care providers, rooms, beds, special equipment, etc.) and/or impending patient transfers.

FIG. 5B is a block diagram illustrating the settings region 310 of the example user interface of a real time command center staff display 300 of FIG. 5A according to an embodiment of the invention. In the illustrated embodiment, the settings region 301 includes a section for a hospital setting 20, a section for an urgent care setting 30 and a section for an other care setting 60.

The section for the hospital setting 20 comprises a plurality of portions including a first portion for an emergency department 317 and a second portion for an urgent care department 274. These departments are administratively part of the hospital setting 20. The separate patient queues each include a prioritized list of patients.

The section for the urgent care setting 30 comprises a plurality of portions including a first portion for an urgent care department 028 and a second portion for an urgent care department 183 and a third portion for an urgent care department 202. The three urgent care departments are administratively part of the same urgent care setting 30. The separate urgent care patient queues each include a prioritized list of patients.

The section for the other care setting 60 comprises a plurality of portions including a first portion for a mobile transport vehicle 011 and a second portion for a long term care facility 014. The mobile transport vehicle setting and the long term care facility setting are administratively separate. In one embodiment, each patient listed in the mobile transport vehicle 011 portion is presently being transported in a separate mobile transport vehicle (e.g., ambulance, helicopter, etc.). Advantageously, the separate patient queues each include a prioritized list of patients.

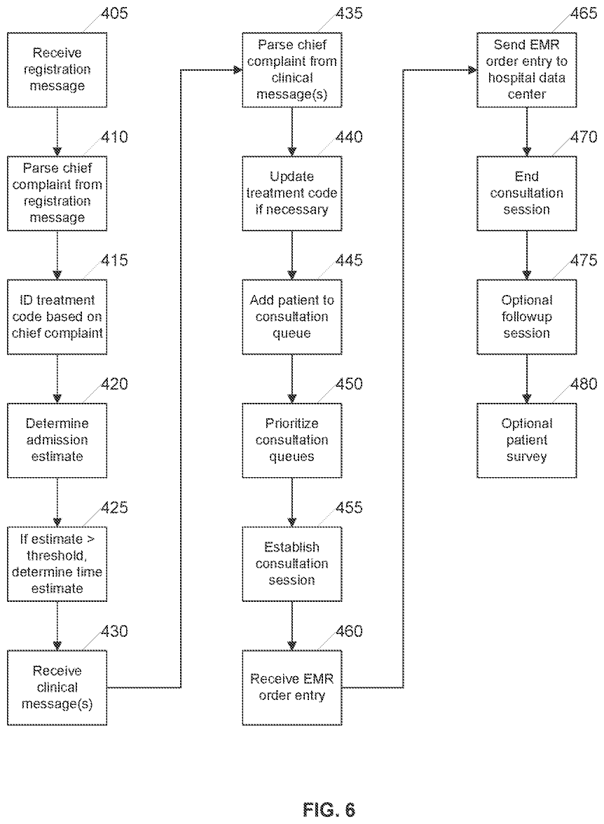

FIG. 6 is a flow diagram illustrating an example process for conducting a consultation session between command center staff and a patient according to an embodiment of the invention. In one embodiment, the illustrated process may be carried out by a system such as previously described with respect to FIGS. 1-5. The present embodiment will be described with respect to HL7 messages, but alternative clinical messaging protocols may also be employed. Initially, in step 405 the system receives an ADT registration message and subsequently parses the ADT registration message in step 410 to identify a chief complaint for the patient. The ADT registration message is a form of HL7 message and is typically used when a patient initially checks into a health care facility. Next, in step 415 the system uses the chief complaint to identify a treatment code for the patient and based on the treatment code, the system determines an initial admission estimate in step 420. In one embodiment, the initial admission estimate may be determined as a percentage. Next, in step 425 if the initial admission estimate exceeds a predetermined threshold, e.g., 89%, then the system additionally determines an estimated admission time. The estimated admission time may be determined as a time of day (e.g., 14:50) or as an increment to the current time of day (e.g., 2 hours and 30 minutes).

Next, in step 430 the system receives one or more additional HL7 messages associated with the patient and parses any chief complaint information from the additional HL7 messages in step 435. For example, a patient may be initially seen by a triage clerk in an emergency department who initiates sending the ADT registration message and then be subsequently seen by a local care provider (i.e., triage personnel) who initiates sending one or more additional HL7 messages that may include additional chief complaint information and other clinical observations such as a patient's vital signs. Next, in step 440, the system updates the previously determined treatment code for the patient, if necessary. In an alternative embodiment, the system may add an additional treatment code corresponding to the subsequent HL7 message such that the particular patient has two or more treatment codes corresponding to two or more HL7 messages. Advantageously, this allows the command center staff to review any changes to the patient's chief complaint or other clinical observations over time.

Next in step 445 the system adds the patient to the specific department's patient consultation queue and prioritizes the patient amongst the patients at the same department. In one embodiment, the patient is automatically added to the consultation queue and prioritized. In such an embodiment, the patient can be removed from the patient consultation queue at any time upon receipt of an instruction from the local care provider device to remove the patient from the patient consultation queue. It may be desirable to remove a patient from the patient consultation queue because the patient may be either too sick or not sick enough to be seen by remote command center staff. In an alternative embodiment, the patient is added to the patient consultation queue in response to receiving a request from the local care provider device to add the patient to the consultation queue.

Subsequently in step 450, the system prioritizes the patient amongst all patients in all settings being monitored by the system. In one embodiment, the system presents a combined prioritization queue in the combined prioritization patient queue section 310 of the command center staff display 300.

Next, in step 455, the system establishes a digital consultation session between the command center staff device at the command center and the local care provider device or patient device (or other device) at the department where the patient is located. In one embodiment, the digital consultation session may be a three way session between the command center staff device and the local care provider device and the patient device. The local care provider device may optionally only participate in the three way digital consultation session by way of a text chat interface with the command center staff device to facilitate private communication between the command center staff and the local care provider while the local care provider is physically in the presence of the patient. Accordingly, the digital consultation session may include audio, video, text, or other data elements such as applications that are presented on the display of one of the command center staff device, the local care provider device, the patient device, or other device. The digital consultation session may also provide audio data to audio output devices in proximity to one or more of the various display devices.

During or after the digital consultation session, in step 460 the system receives EMR order entry information from the command center staff device at the command center and in step 465 the system provides the received order entry information to the data center device at the hospital data center. In step 470, the system ends the session between the command center staff device and the local care provider device or patient device (or other device). The ending of the session in step 470 may take place before or after steps 460 and 465. In one embodiment, the system may be configured to establish an optional digital consultation session in step 475 between the command center staff device at the command center and a local care provider device after ending a digital consultation session between command center staff and a patient for routine follow up and additional instructions from the command center staff to the local care provider or to facilitate review by the command center staff of diagnostic data and entry of new or revised testing and/or treatment orders. Additionally, the system may be configured to establish an optional patient survey session in step 480 with the patient to gauge patient satisfaction with the acute medical care facility visit.

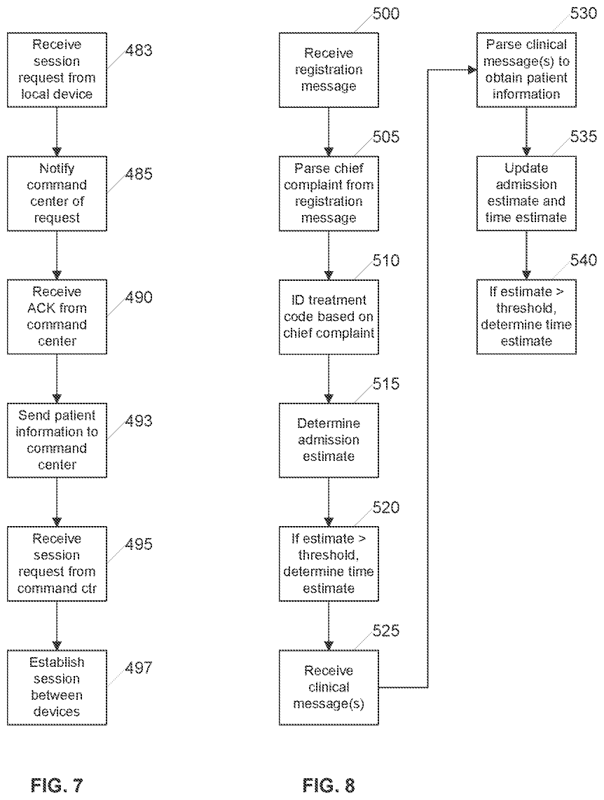

FIG. 7 is a flow diagram illustrating an example process for establishing a digital consultation session according to an embodiment of the invention. In one embodiment, the illustrated process may be carried out by a system such as previously described with respect to FIGS. 1-5. Initially, in step 483 the system receives a consultation request from a device located at one of the various local care provider departments of a setting being managed by the system, e.g., a patient device or a local care provider device at an emergency department. In one embodiment, the consultation request is received from the local care provider device with an instruction to establish the consultation session with the patient device only or the patient device and the local care provider device simultaneously. In response to receiving the consultation request, the system notifies the command center of the request in step 485. The notice may cause the name of the patient in one or more patient queues of the user interface on the display of the command center device to be emphasized or otherwise visually distinguished to indicate that a consultation request for the patient has been received. Subsequent to sending the notice in step 485, the system receives a corresponding acknowledgement from the command center to confirm that a consultation session is imminent. In one embodiment, the acknowledgement includes the name of the command center staff and the system provides the command center staff person name and title to the local care provider and/or patient device(s) to allow presentation on the display of the local care provider and/or patient device(s) a courtesy message stating that “Dr. Smith will be with the patient shortly” or something similar. Biographical information about the doctor may include a picture may also be provided in the acknowledgement and presented on the display of the local care provider and/or patient device(s). In response to receiving the acknowledgement, in step 493, the system sends patient information to the command center. Patient information includes the patient EMR, suggested testing and treatment protocols, links to relevant information or reference manuals, contact information, diagnostic data (if available), and the like. Such information can advantageously be presented on a display at the command center for review by command center staff prior to commencing the digital consultation session.

Next, in step 495, the system receives from the command center a session request corresponding to the consultation request received from the local care provider department. The system then establishes a digital consultation session between the one or more local care provider and/or patient devices and the command center device.

FIG. 8 is a flow diagram illustrating an example process for determining a hospital admission estimate for a patient according to an embodiment of the invention. In one embodiment, the illustrated process may be carried out by a system such as previously described with respect to FIGS. 1-5. Initially, steps 500-520 are similar to and were previously described with respect to steps 405-425 of FIG. 6 and will not be separately discussed with respect to FIG. 8. In step 525, the system receives one or more additional HL7 messages associated with the patient and parses additional patient information from the additional HL7 messages in step 530. Such additional information may include chief complaint information, vital signs, laboratory information, medical imaging information, EMR information such as patient family and medical history, testing and diagnostic orders, lab results, imaging results, EKG results, previous patient encounter information, previous diagnoses, notes and other information related to a patient and/or the patient's history and the patient's current medical condition and/or needs, billing information, or any other patient related information in an HL7 message (or other type of message). Next, in step 535 the system updates the admission estimate based on the additional information parsed from the additional HL7 messages. For example, if X-RAY information indicates a severe fracture requiring surgery, the admission estimate may be updated to 100%. Finally, in step 540 if the updated admission estimate exceeds the predetermined threshold, e.g., 89%, then the system determines an initial estimated admission time (e.g., if the prior admission estimate was below the predetermined threshold) or updates the previously determined estimated admission time. As previously discussed, the updated estimated admission time may be determined as a time of day (e.g., 14:50) or as an increment to the current time of day (e.g., 2 hours and 30 minutes from the present time).

### Example Embodiment

An example embodiment will now be described to illustrate the operation of the system and underscore certain advantages and capabilities provided by use of the system that were previously not possible.

Referring back to FIG. 1, in the example embodiment a command center is staffed with a plurality of command center staff and at least one or more of the command center staff is credentialed and privileged at one or more medical care settings. Notably, the various command center staff are not necessarily credentialed and privileged at the same medical care settings. Each of a plurality of command center staff accesses a command center staff device having a user interface (e.g., display monitor, keyboard, mouse, camera, microphone, speaker(s), etc.) and provides login credentials for each of the various medical care settings at which the command center staff is credentialed and privileged. The login credentials are received at a control device (e.g., located at a server center) and the control device automatically accesses a data center device via a data communication network at each of the various medical care settings at which the respective command center staff is credentialed and privileged. The control device provides each medical care setting data center device with the appropriate login credentials for the command center staff that are credentialed and privileged at the respective medical care setting. Advantageously, the control center device automatically maintains an active session for each command center staff with each respective medical care setting during the shift of the respective command center staff at the command center. The login credentials may advantageously provide the control device with access to both an EMR system and a Computerized Physician Order Entry (“CPOE”) system at each respective medical care setting for which the respective command center staff is credentialed and privileged.

The control device also receives patient information from each of a plurality of medical care settings and stores and aggregates the patient information into prioritized patient queues for each medical setting and the control device also creates an aggregate patient queue combining all patients from all medical care settings and prioritizes the aggregate patient queue based on an analysis of individual patient information such as vital signs and chief complaint. The control device may also adjust the priority of any individual patient in each prioritized patient queue (aggregate and single care setting) based on a prioritization message received from a medical care setting.

The control device presents a user interface on the display monitor of a command center staff device and the user interface includes the prioritized medical care setting patient queues and also the aggregate prioritized patient queue. In each prioritized patient queue, certain patient information is included. For example age, gender, vital signs and other related information for each patient may be presented in association with each patient identifier in a patient queue. Importantly, each command center staff device is associated with the login credentials provided by the command center staff upon initial access and subsequently, all information presented on the display monitor of that specific command center staff device is filtered such that only patient information from those medical care settings at which the corresponding command center staff is credentialed and privileged is provided to the specific command center staff device.

Accordingly, a command center staff at a command center staff device is presented with a user interface populated with prioritized patient queues for each of a plurality of medical settings at which the command center staff is credentialed and privileged. The user interface is also populated with an aggregate prioritized patient queue across a plurality of medical settings at which the command center staff is credentialed and privileged. Advantageously, when the control device receives a selection of a specific patient via the user interface of the command center staff device, the control device populates the user interface with at least the EMR of the selected patient. The control device obtains the EMR from a data storage area associated with the control device and eliminates the inefficiency associated with accessing the EMR from the medical care setting at which the patient is physically located. These inefficiencies include but are not limited to providing the appropriate login credentials to a data center device associated with the medical care setting, receiving significant amounts of medical record data (e.g., data including multiple gigabytes of medical imaging data) via a data communication network transmission, and the time and potential for error introduced by manually navigating to the patient record associated with the specific patient in the user interface. The consolidated presentation of patients from multiple medical care settings is particularly advantageous because it is not possible for a physician to be physically present at multiple medical care settings simultaneously. Consequently, a physician is not physically able to prioritize patients across multiple care settings and choose to consult with the highest priority patient, regardless of the medical care setting at which each patient is physically located.

However, the user interface of the command center staff device provides the command center staff with precisely this ability. Accordingly, the control device obtains and locally stores patient information for a plurality of patients that are associated with a plurality of medical care settings. The locally stored patient information is subsequently used for analysis by the control device (e.g., to prioritize patient queues, to automatically obtain recommended treatment information, etc.) and for presentation on the display monitor of a command center staff device, just to name two uses of the locally stored patient information. The control device also analyzes information corresponding to each patient to identify a chief complaint for the specific patient and the control device also obtains treatment information related to the specific patient's chief complaint, e.g., testing protocols, treatment protocols, and medical references about certain conditions associated with the chief complaint. This treatment information is also stored and/or accessed by the control device and is advantageously populated into the command center staff device user interface along with the EMR record for the specific patient after the control device receives a selection of the specific patient from the command center staff device.

The user interface is also populated with a session interface to allow the command center staff to consult with the patient via an audio/visual/text session. Additionally, the control device populates the user interface with an EMR data region and a CPOE (e.g., order entry) region to allow the command center staff to review the patient's medical history and order tests and laboratory diagnostics to be conducted at the medical care setting or elsewhere. In one embodiment, the control device populates the user interface with a uniform order entry format for simplified order entry by command center staff and upon receipt of an order via the user interface for a certain patient, the control device translates the uniform order entry format data into medical care setting specific format data and provides the medical care setting specific format data to a data center device associated with the corresponding medical care setting at which the certain patient is physically located. In an alternative embodiment, the control device populates the user interface with the order entry format of the respective order entry system.

Advantageously, the user interface is populated with an aggregate patient queue of patients across a plurality of medical care settings and the user interface is also populated with a recommended prioritization for each patient in the aggregate patient queue. Command center staff can rapidly access information about individual patients and review individual patient vital signs, chief complaint, EMR data and related treatment information corresponding to the patient. Advantageously, command center staff may review information about a plurality of patients at a plurality of separate medical care settings before selecting to consult with a specific patient. When the control device receives a selection to consult with a specific patient, the control device establishes a telemedicine session (e.g., audio, video, text, etc.) between the command center staff device and a patient device in proximity of the patient. During and after the telemedicine session, the command center staff is able to enter orders and prescriptions, etc. via the CPOE region of the user interface. Upon completion of the telemedicine session and completion of any CPOE orders and/or notes into the patient's EMR, the command center staff may immediately begin accessing and reviewing information about additional patients in the aggregate patient queue or an individual medical setting patient queue and rapidly select a next patient to consult with. In this fashion, the command center staff can be utilized more efficiently than a physician that is physically resident at an isolated individual medical care setting.

FIG. 9 is a block diagram illustrating an example wired or wireless system 550 that may be used in connection with various embodiments described herein. For example the system 550 may be used as or in conjunction with a patient device, requestor device, provider device, control device, data center device, server device, display device or any other processor implemented device as previously described with respect to FIGS. 1-5B. The system 550 can be a conventional personal computer, computer server, personal digital assistant, smart phone, tablet computer, or any other processor enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include an internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown) that are executable by processor 560.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. A method comprising using at least one hardware processor of a server system to:
generate a graphical user interface comprising a single screen that is logically divided into a plurality of sections, wherein the plurality of sections comprises a queue section and an electronic medical record (EMR) section that are simultaneously displayed in the single screen, wherein the queue section comprises at least one prioritized patient queue comprising a list of patients from at least one medical care setting;
in response to selection of a patient from the list of patients in the queue section, retrieve EMR information associated with the selected patient, and populate the EMR section with at least a portion of the EMR information;
in response to a user operation, initiate a digital consultation session with a remote device associated with the selected patient over at least one network; and,
in response to an order that is input by a user, initiate an action for treatment of the selected patient in accordance with the order.

2. The method of claim 1, wherein, in response to the selection of the patient from the list of patients in the queue section, the EMR information associated with the selected patient is retrieved from a local data storage area of the server system without requiring communication with any system in the at least one medical care setting.

3. The method of claim 1, wherein the queue section comprises a plurality of prioritized patient queues, wherein each of the plurality of prioritized patient queues comprises a list of patients from at least one medical care setting.

4. The method of claim 3, wherein the plurality of prioritized patient queues comprises:
one or more department-specific patient queues, wherein each of the one or more department-specific patient queues comprises a list of patients from a single department; and
a global patient queue comprising a list of patients across a plurality of departments.

5. The method of claim 1, further comprising using the at least one hardware processor of the server system to:
receive a message from at least one medical setting;
parse the message to extract patient information, wherein the patient information comprises a chief complaint;
rank a first patient represented in the extracted patient information; and
add the first patient to the at least one prioritized patient queue at a position in the at least one prioritized patient queue that is in accordance with the rank of the first patient.

6. The method of claim 5, wherein the message is an admission/discharge/transfer (ADT) message of a Health Level Seven (HL7) standard.

7. The method of claim 5, further comprising using the at least one hardware processor of the server system to calculate an initial admission estimate based on the chief complaint.

8. The method of claim 5, further comprising using the at least one hardware processor of the server system to determine a treatment code based on the chief complaint.

9. The method of claim 8, wherein the determination of the treatment code is based on historical orders for treatment and actual outcomes of treatment.

10. The method of claim 8, further comprising using the at least one hardware processor of the server system to translate the treatment code into a suggested treatment protocol.

11. The method of claim 10, wherein the plurality of sections further comprises a suggested treatment section, and wherein the method further comprises using the at least one hardware processor of the server system to indicate the suggested treatment protocol in the suggested treatment section.

12. The method of claim 10, further comprising using the at least one hardware processor of the server system to:
generate one or more inputs in the single screen of the graphical user interface for selecting the suggested treatment protocol as the order; and,
in response to selection of the suggested treatment protocol as the order, initiate an action for treatment of the selected patient in accordance with the suggested treatment protocol.

13. The method of claim 5, further comprising using the at least one hardware processor of the server system to:
receive one or more additional messages from the at least one medical care setting associated with the first patient; and
adjust the position of the first patient within the at least one prioritized patient queue based on the one or more additional messages.

14. The method of claim 13, further comprising using the at least one hardware processor of the server system to:
record timestamps from the message and the one or more additional messages; and
calculate a measure of time between admission and treatment of the first patient based on the recorded timestamps.

15. The method of claim 14, further comprising using the at least one hardware processor of the server system to construct a chronological timeline of a path traveled by the first patient through the at least one medical care setting based on the recorded timestamps.

16. The method of claim 1, wherein initiating the action for treatment comprises:
translating the order into data that is in a format that is compatible with the at least one medical care setting; and
sending the data over at least one network to a system in the at least one medical care setting.

17. The method of claim 1, wherein the plurality of sections further comprises an EMR order entry section comprising one or more inputs for input of the order, and wherein the EMR order entry section is in a uniform format that is identical across all medical settings.

18. The method of claim 1, further comprising using the at least one hardware processor of the server system to filter the queue section to include only patients from medical care settings for which a user is authorized based on one or more credentials or privileges of the user.

19. A system comprising:
at least one hardware processor; and
one or more software modules configured to, when executed by the at least one hardware processor,
generate a graphical user interface comprising a single screen that is logically divided into a plurality of sections, wherein the plurality of sections comprises a queue section and an electronic medical record (EMR) section that are simultaneously displayed in the single screen, wherein the queue section comprises at least one prioritized patient queue comprising a list of patients from at least one medical care setting,
in response to selection of a patient from the list of patients in the queue section, retrieve EMR information associated with the selected patient, and populate the EMR section with at least a portion of the EMR information,
in response to a user operation, initiate a digital consultation session with a remote device associated with the selected patient over at least one network, and,
in response to an order that is input by a user, initiate an action for treatment of the selected patient in accordance with the order.

20. A non-transitory computer-readable medium having instructions stored thereon, wherein the instructions, when executed by a processor, cause the processor to:
generate a graphical user interface comprising a single screen that is logically divided into a plurality of sections, wherein the plurality of sections comprises a queue section and an electronic medical record (EMR) section that are simultaneously displayed in the single screen, wherein the queue section comprises at least one prioritized patient queue comprising a list of patients from at least one medical care setting;
in response to selection of a patient from the list of patients in the queue section, retrieve EMR information associated with the selected patient, and populate the EMR section with at least a portion of the EMR information;
in response to a user operation, initiate a digital consultation session with a remote device associated with the selected patient over at least one network; and,
in response to an order that is input by a user, initiate an action for treatment of the selected patient in accordance with the order.
